(19) United States
(12) Patent Application Publication
(10) Pub. No.: US 2012/0179270 A1
(43) Pub. Date: Jul. 12, 2012

(54) LOW STRESS MULTIPLE FIXATION ACETABULAR COMPONENT

(76) Inventors: Russell Nevins, Las Vegas, NV (US); Edward J. McPherson, Los Angeles, CA (US); Timothy McTighe, Chagrin Falls, OH (US); Declan Brazil, Chatswood (AU)

(21) Appl. No.: 13/346,687

(22) Filed: Jan. 9, 2012

Related U.S. Application Data
(63) Continuation-in-part of application No. 13/189,448, filed on Jul. 22, 2011, now abandoned.
Application No. 13/189,448 is a continuation of application No. 13/045,447, filed on Mar. 10, 2011, now abandoned.
(60) Provisional application No. 61/312,650, filed on Mar. 10, 2010.

(51) Int. Cl. A61F 2/34

(52) U.S. Cl. 623/22.35; 623/22.39

(57) ABSTRACT

The disclosure relates to revision hip surgery or total hip arthroplasty situations in which it is difficult for a surgeon to secure or fix an acetabular cup or shell to the bone due, for whatever reason, to bone loss or other bone deficiency. The system may comprise a plurality of fasteners and an acetabular cup or shell having a plurality of openings to thereby allow the plurality of fasteners to be inserted at diverging angles to create multiple fixation points to distribute load and secure the shell to available bone. The system may also include a bearing insert for receiving a femoral head component and a plurality of spacers for creating a cement mantle.

### CROSS-REFERENCE TO RELATED APPLICATIONS

This application is a continuation-in-part of co-pending U.S. patent application Ser. No. 13/189,448, filed Jul. 22, 2011, entitled “LOW STRESS MULTIPLE FIXATION ACETABULAR COMPONENT,” which itself is a continuation of and claims priority to U.S. patent application Ser. No. 13/045,447, filed Mar. 10, 2011, entitled “LOW STRESS MULTIPLE FIXATION ACETABULAR COMPONENT,” which itself claims the benefit of U.S. Provisional Application No. 61/312,650, filed Mar. 10, 2010, all of which are hereby incorporated by reference herein in their entireties, including but not limited to those portions that specifically appear hereinafter, the incorporation by reference being made with the following exception: In the event that any portion of any of the above-referenced applications is inconsistent with this application, this application supercedes said above-referenced applications.

### STATEMENT REGARDING FEDERALLY SPONSORED RESEARCH OR DEVELOPMENT

Not Applicable.

### BACKGROUND

1. The Field of the Disclosure.

The disclosure relates generally to artificial joint prostheses and orthopedic implants, and more particularly, but not necessarily entirely, to acetabular prostheses used in hip joint replacement surgeries and revision surgeries.

2. Description of Related Art

Total hip joint replacements comprise a femoral component and an acetabular component. The femoral component is implanted in the patient's femur and the acetabular component is implanted in the acetabular cavity in the patient's pelvis. The normal acetabular cavity is generally spherically shaped. Accordingly, as illustrated in FIG. 1, during initial hip joint replacement, a spherically-shaped cavity 10 is prepared in the pelvis 14 for the great majority of patients, and the outer surface of the acetabular prosthesis 12 has a generally spherical shape to fit in this cavity.

Over the past several decades, millions of hip joint replacements have been performed. As time has progressed, a significant number of these implants have failed due to aseptic loosening of either or both of the femoral or acetabular components. In the case of the acetabulum, a loose prosthesis often erodes bone in the direction of the applied forces during use, i.e., in the superior and posterior directions. This is particularly true where the loosened prosthesis had been implanted using bone cement. As a result, during a revision hip joint replacement, the surgeon often finds that the once spherical cavity in the pelvis has become elongated in some manner as shown at 16 in FIG. 2. Also, in some cases, elongated acetabular cavities or other bony defects may be found for patients undergoing their first hip joint replacement.

The classical way to deal with the bone defects, such as an elongated acetabular cavity, has been to fill a portion of the cavity with a bone graft to recreate, as best as possible, a spherical cavity in its normal location. As is evident, this approach suffers from a variety of problems, including availability of bone for the bone graft, mechanical difficulties of securing the bone graft to the host bone, failure of the bone graft to provide and maintain long term mechanical support for the prosthesis, and the hazard of the spread of certain infectious diseases.

Bony defects may be treated by using jumbo acetabular cups, oblong acetabular cups, modular augments that attach the acetabular cup to the bone or other methods currently available.

It will be appreciated that a longitudinal axis of the acetabulum has a natural angle with respect to the midline of a patient. For maximum fixation or containment, the outer shell of the acetabular component of a hip implant may be inserted into the acetabulum at an angle of about 50 degrees to about 60 degrees. Surgeons often position the shell at an angle of about 55 degrees. For joint stability, the preferred angle of the face of the bearing element in the coronal plane is on the order of 45 degrees or less. Moreover, the greater the elongation of the cavity, the greater the departure from the preferred orientation. In terms of function, such a geometry means that the patient will have a significantly higher likelihood of dislocation during use.

In addition to the orientation in the coronal plane, the functionality of the prosthesis is also affected by the orientation of the face of the bearing element in the transverse plane. In this case, the angle between the face of the bearing element and the sagittal plane is on the order of about 15 degrees to about 20 degrees anteverted (note that the amount of anteversion increases as the angle increases.)

Due to the curvature of the pelvis and the usual direction of erosion and elongation, the prior art oblong acetabular prostheses tend to assume an orientation which is less anteverted or, in some cases, may even be retroverted. The magnitude of this problem also becomes greater as the elongation of the cavity becomes greater. Again, in terms of function, orientations which are less anteverted or are retroverted mean that the patient will have a higher likelihood of dislocation during use.

Despite the advantages ofknown hemispherically shaped acetabular cup systems and devices, improvements are still being sought. However, the prior art is characterized by several disadvantages that may be addressed by the disclosure. The disclosure minimizes, and in some aspects eliminates, these failures, and other problems, by utilizing the methods and structural features described herein.

The features and advantages of the disclosure will be set forth in the description which follows, and in part will be apparent from the description, or may be learned by the practice of the disclosure without undue experimentation. The features and advantages of the disclosure may be realized and obtained by means of the instruments and combinations particularly pointed out herein.

### BRIEF DESCRIPTION OF THE DRAWINGS

The features and advantages of the disclosure will become apparent from a consideration of the subsequent detailed description presented in connection with the accompanying drawings in which:

FIG. 1 is a side, cross-sectional view showing the implantation of a hemispherically-shaped acetabular cup or shell in an acetabular cavity of a patient's pelvis;

FIG. 2 is a side, cross-sectional view showing an acetabular cavity of a patient's pelvis, which has become elongated;

FIG. 3 is a bottom view of an embodiment of an acetabular cup or shell implanted into a patient's pelvis and made in accordance with the principles of the disclosure;

FIG. 4 is a perspective view of an embodiment of an acetabular cup or shell shown in combination with a plurality of fasteners and made in accordance with the principles of the disclosure;

FIG. 4A is a cross-sectional view of an embodiment of an acetabular cup or shell shown in combination with a fastener implanted into a patient's pelvis and made in accordance with the principles of the disclosure;

FIG. 5 is a bottom perspective view of an embodiment of a bearing insert made in accordance with the principles of the disclosure;

FIG. 6 is a bottom perspective view of an embodiment of a bearing insert and an acetabular cup or shell made in accordance with the principles of the disclosure;

FIG. 7 is a side view of an embodiment of a bearing insert with bone cement applied to an external surface thereof and made in accordance with the principles of the disclosure;

FIG. 8 is a bottom perspective view of an embodiment of a bearing insert inserted into an acetabular cup or shell and made in accordance with the principles of the disclosure;

FIG. 9 is a perspective view of an embodiment of a bearing insert inserted into an acetabular cup or shell and made in accordance with the principles of the disclosure;

FIG. 10 is an illustration of an x-ray image of an embodiment of the acetabular cup or shell as described herein fastened to a patient's acetabulum with a plurality of fasteners;

FIG. 11 is a bottom view of an embodiment of an acetabular cup or shell made in accordance with the principles of the disclosure;

FIG. 12 is a side view of the embodiment of the acetabular cup or shell of FIG. 11;

FIG. 13 is a side, cross-sectional view of the acetabular cup or shell taken along section A-A of FIG. 12;

FIG. 14 is another side, cross-sectional view of the acetabular cup or shell taken along section A-A of FIG. 12 illustrating the coating details of the acetabular cup or shell;

FIG. 15 is an enlarged, side view of an opening in the acetabular cup or shell as shown in Detail C of FIG. 13;

FIG. 16 is a side, partial cross-sectional view of the acetabular cup or shell taken along section D-D of FIG. 11;

FIG. 17 is an enlarged, side view of an opening in the acetabular cup or shell as shown in Detail E of FIG. 13;

FIG. 18 is a perspective view of an embodiment of an acetabular cup or shell of the disclosure;

FIG. 19 is a bottom view of the embodiment of the acetabular cup or shell illustrated in FIG. 18;

FIG. 20 is a side, cross-sectional view of the acetabular cup or shell taken along section F-F of FIG. 19;

FIG. 21 is a side, cross-sectional view of the acetabular cup or shell taken along section F-F of FIG. 19 illustrating the coating details of the acetabular cup or shell;

FIG. 22 is an enlarged, side view of an opening in the acetabular cup or shell as shown in Detail H of FIG. 21;

FIG. 23 is a top view of an embodiment of an acetabular cup or shell made in accordance with the principles of the disclosure;

FIG. 24 is a cross-sectional view of the acetabular cup or shell taken along section A-A of FIG. 23 and made in accordance with the principles of the disclosure;

FIG. 24A is a side, cross-sectional view of an embodiment of an acetabular cup or shell illustrating angles of the acetabular cup or shell and made in accordance with the principles of the disclosure;

FIG. 24B is a side, cross-sectional view of an embodiment of an acetabular cup or shell illustrating angles of the acetabular cup or shell and made in accordance with the principles of the disclosure;

FIG. 24C is a side, cross-sectional view of an embodiment of an acetabular cup or shell illustrating angles of the acetabular cup or shell and made in accordance with the principles of the disclosure;

FIG. 24D is a side, cross-sectional view of an embodiment of an acetabular cup or shell illustrating angles of the acetabular cup or shell and made in accordance with the principles of the disclosure;

FIG. 24E is a side, cross-sectional view of an embodiment of an acetabular cup or shell made in accordance with the principles of the disclosure;

FIG. 25 is a side view of the acetabular cup or shell of FIG. 23 made in accordance with the principles of the disclosure;

FIG. 26 is a perspective view of the acetabular cup or shell taken from the perspective of B-B in FIG. 24;

FIG. 27 is an enlarged, side view of a figure eight shaped opening in the acetabular cup or shell as shown in Detail C of FIG. 26;

FIG. 28 is a perspective view of an embodiment of an assembly of the disclosure, including an acetabular cup or shell, a bearing insert, and a plurality of fasteners made in accordance with the principles of the disclosure;

FIG. 29 is an enlarged, side view of an inner surface of the acetabular cup or shell as shown in Detail B of FIG. 28; and

FIGS. 30 through 35 illustrate bottom views ofvarious embodiments of an acetabular cup or shell with openings in various locations and numbers made in accordance with the principles of the disclosure.

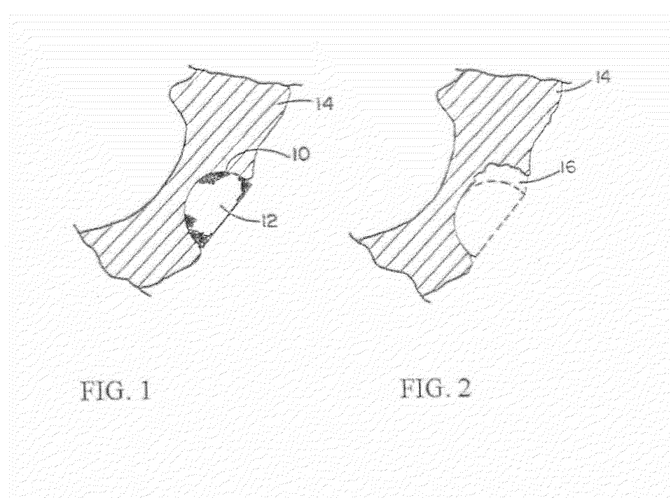

### DETAILED DESCRIPTION

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure.

Before the system, method and devices for improving stability and fixation in an acetabular cup when there is less than optimal amount of bone to secure the acetabular cup to the acetabulum, whether used in a revision or total hip arthroplasty, are disclosed and described, it is to be understood that this disclosure is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the disclosure will be limited only by the appended claims, if any, and equivalents thereof.

In describing and claiming the disclosure, the following terminology will be used in accordance with the definitions set out below.

It must be noted that, as used in this specification and the appended claims, the singular forms “a,” “an,” and “the” include plural referents unless the context clearly dictates otherwise.

As used herein, the terms “comprising,” “including,” “containing,” “characterized by,” and grammatical equivalents thereof are inclusive or open-ended terms that do not exclude additional, unrecited elements or method steps.

As used herein, the term “proximal” shall refer broadly to the concept of a nearest portion or a portion nearest the midline.

As used herein, the term “distal” shall generally refer to the opposite of proximal, and thus to the concept of a further portion, or a portion furthest from the midline, depending upon the context.

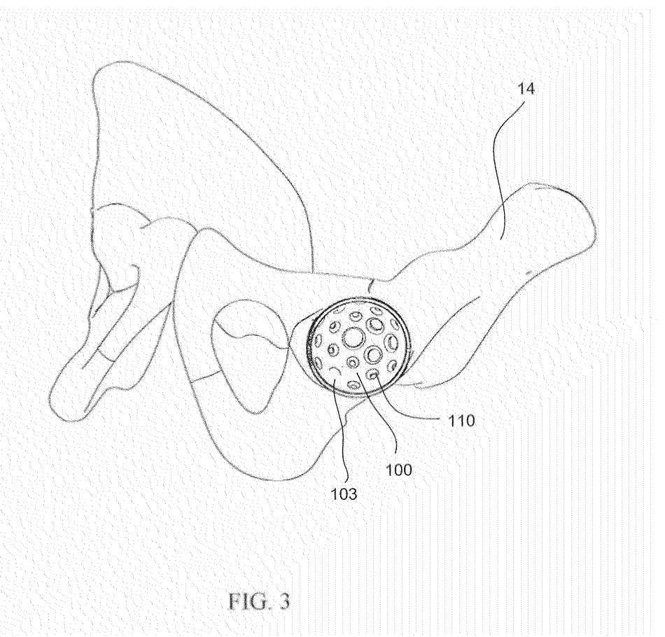

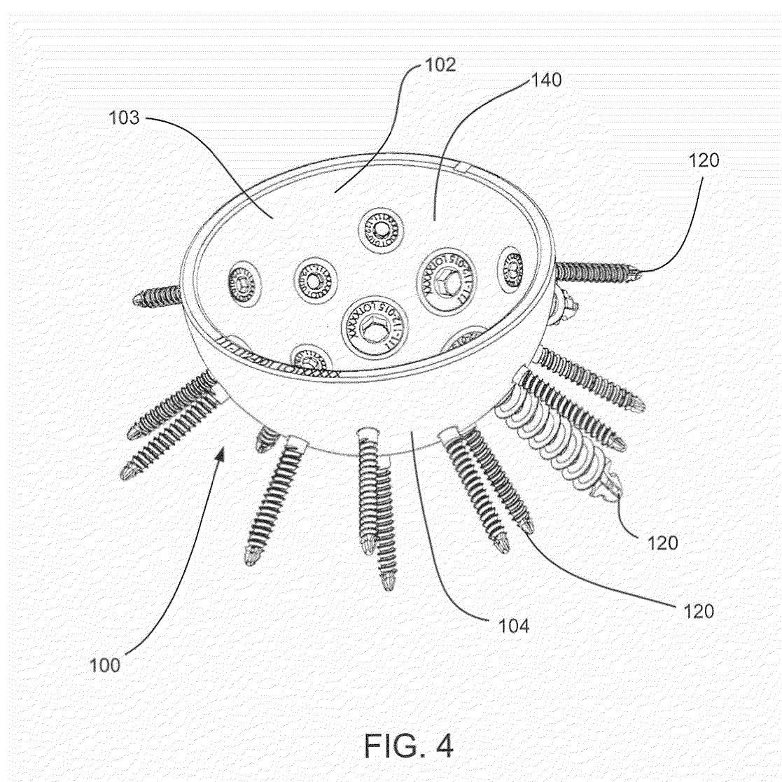

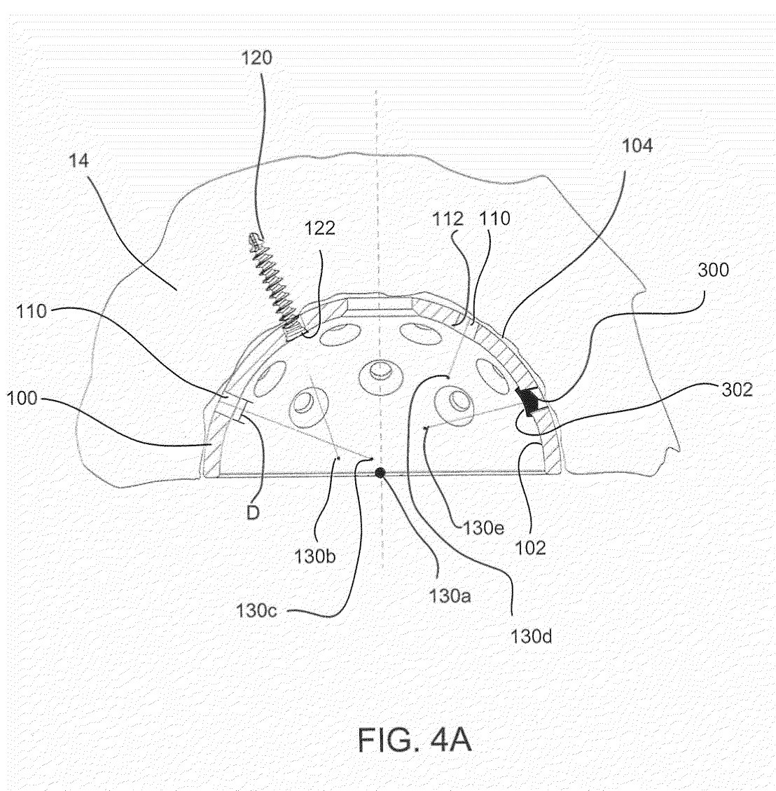

Referring now to FIGS. 3 and 4, there is illustrated an embodiment of an acetabular cup or shell 100 made in accordance with the principles of the disclosure. The acetabular cup 100 may be hemispherically shaped or may be any other shape to correspond with the shape of the acetabulum, whether the acetabulum is hemispherically shaped or otherwise deformed in some respect. It will be appreciated that the acetabulum may comprise a defect caused by forces placed thereon or a lack of force placed thereon. One of the most common acetabular cup shapes is the hemispherical cup. Surgeons are well acquainted with this type of hemispherical cup. However, in some cases, such as a revision or a total hip arthroplasty with a defect in the bone, where there is not enough bone to create a tight press fit or otherwise secure the acetabular cup to the bone, another option must be used other than the traditional hemispherical cup.

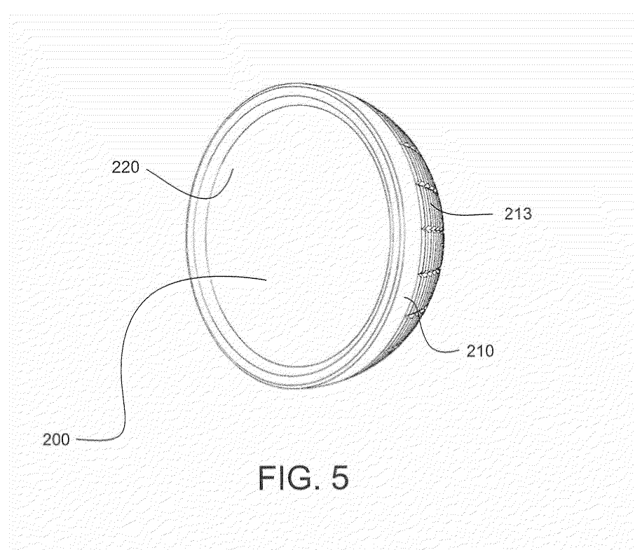

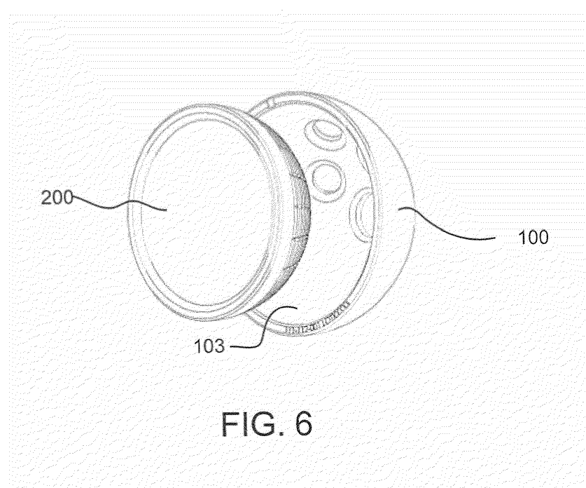

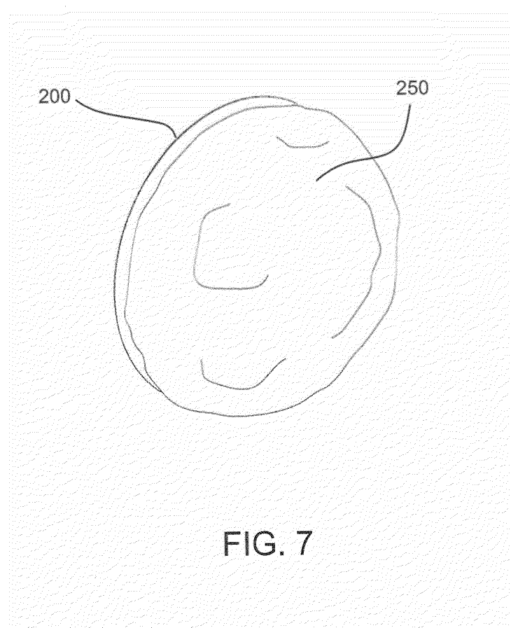

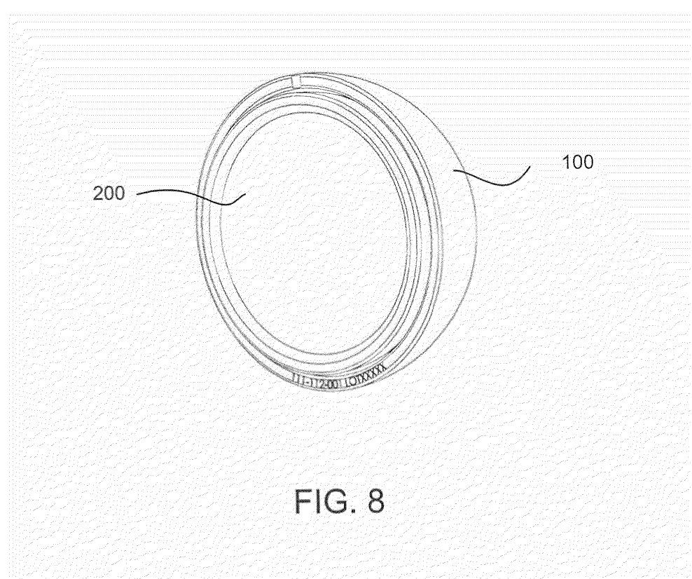

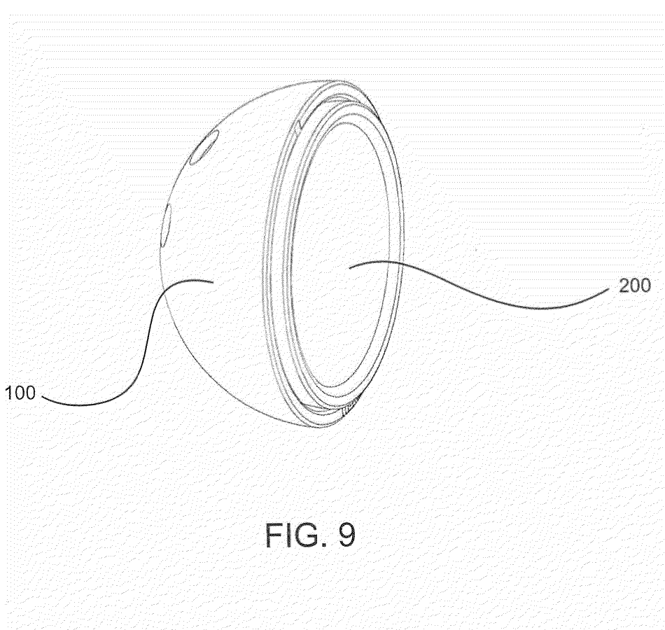

This disclosure relates to those situations in which it is difficult for a surgeon to secure or fix the acetabular cup or shell 100 to the bone due, for whatever reason, to bone loss or other defect. Accordingly, an embodiment of a system for securing an acetabular cup or shell 100 to the acetabulum may comprise an acetabular cup or shell 100 itself as illustrated in FIGS. 3 and 4, a bearing insert 200 (illustrated best in FIGS. 5-10) for receiving a femoral head component and a plurality of spacers 300 for creating a cement mantle as described below in more detail.

Referring to FIGS. 3 through 4A, the cup or shell 100 may comprise a plurality of openings or fenestrations 110 for allowing a fastener 120 to pass therethrough and into the bone. The cup or shell 100 may comprise a first, inner surface 102 and a second, outer surface 104.

The first surface 102 of the cup or shell 100 may comprise a face 103 that has a finish that is porous or that has a matte finish to permit a bonding agent, such as bone cement, to bond thereto. It will be appreciated that the first surface 102 may be comprised of any non-smooth finish or a rough finish for receiving bone cement without departing from the scope of the disclosure. For example, the first surface 102 may comprise a hydroxyapatite (HA) finish, which is a basic mineral containing calcium and phosphorus and contributes to the strength of bone. Hydroxyapatite may be a beneficial material for increasing the interdigitation between the bonding agent and the first surface 102, which may permit increased interdigitation between components that may be bonded together with a bonding agent since hydroxyapatite is a component of bone tissue.

The openings 110 may be formed through the first and second surfaces 102 and 104. It will be appreciated that while the number of openings 110 need not be an exact number, there may be between about thirty and sixty openings 110. Further, the plurality of openings 110 may be located in a pattern across the entire surface of the acetabular cup or shell 100. However, it will be appreciated that the plurality of openings 110 may also be randomly positioned across the entire surface of the acetabular cup or shell 100.

Each opening 110 may be defined by a sidewall 112. Each opening 110 may have the same or a different size diameter “D” that may range from very small to very large and may correspond with the diameter of a corresponding fastener 120. The diameters “D” of the openings 110 may be between a range of about 2.7 millimeters to about 6.5 millimeters to accommodate corresponding fasteners 120. It will be appreciated that the diameter “D” of the openings 110 may be any size, such that the diameters “D” correspond directly with the diameter size of the fastener 120 to be inserted therein, such that the diameter size may be larger or smaller than the diameters that fall within the above range.

Referring now to FIG. 4A, each of the sidewalls 112 may comprise or may be formed at an angle, which may be the same as or differ from other closely located or situated openings 110. The openings 110 may comprise multiple origins 130 that vary, in which case the sidewalls 112 will be formed at varying angles. In other words, the sidewalls 112 may not all be normal to the inner surface 102 of the cup 100. Alternatively, the plurality of openings 110 may comprise a single origin 130, in which case the openings may be formed or oriented normal to the surface of the acetabular cup or shell 100. Each of the openings 110 may comprise a different origin. It will be appreciated that the origins may fall within a predetermined area that may extend outwardly from the origin 130a of the acetabular cup 100.

The varying angles of each of the sidewalls 112 defining the openings 110 creates the ability for a surgeon to insert fasteners 120 into bony areas at any desired angle to cause fixation to the acetabulum. Further, these varying angles may be divergent so as to create a series of diverging angles. When a series of fasteners 120 is located in a corresponding series of openings 110, i.e., in an area where there is bone to accept or receive fasteners 120, the fasteners 120 are then secured in the bone at diverging angles. The result of the diverging angles at which the fasteners 120 are inserted is maximum fixation and security within the bone.

Further, because the openings 110 may be located across the entire surface of the acetabular cup or shell 100, there are multiple or a plurality of insertion points, such that fasteners 120 may be inserted into any location where there is adequate bone. The multiple insertion points for the fasteners 120 and the divergent angles (created by the sidewalls 112 defining openings 110) at which the fasteners 120 may be inserted into the bone allows the fasteners 120 to adequately grip the bone at various and multiple locations. The result of the gripping is that forces and stresses experienced in the hip joint may be spread over a larger area of the pelvic bone, thus providing maximum fixation and securement of the shell or cup 100.

It will be appreciated that the fasteners 120 may be screws, nails, pins, or any other fastener that may be known or that may become known in the future without departing from the scope of the disclosure. The fasteners 120 may correspond in diameter to the diameters “D” of the openings 110 in the acetabular cup or shell 100. The fasteners 120 may comprise relatively small diameters as well as other more traditional sizes. For example, the fasteners may have a 2.7 millimeter diameter and may be about twenty to thirty-five millimeters in length or the diameters may be larger and more traditional, such as a 6.5 millimeter diameter with a length between about forty to forty-five millimeters in length.

In an embodiment, the plurality of fasteners that may be inserted into the bone may comprise an average diameter that is between a range of about three millimeters to about five millimeters and an average fastener length that is between a range of about twenty-five millimeters to about thirty-five millimeters in length.

It will be appreciated that in an embodiment, the shell may comprise a certain number of openings or throughbores per concentration area of the shell. That certain number of openings may be uniform across the entire outer surface of the shell. As used herein the phrase “concentration area” refers to a portion of the total surface area of the outer surface of the shell measured in millimeters squared. In an embodiment, the plurality of openings may comprise about five to about fifteen openings per concentration area of the outer surface of the shell, which per unit surface area may be between a range of about eighty square millimeters to about one-hundred and seventy-five square millimeters.

It will be appreciated that the use of a plurality or multiple small diameter fasteners 120 may operate to spread the load over a larger area from the acetabular cup or shell 100 to the bone and thereby reduce stress concentration in any one area. For example, in an area where bone contact with the acetabular cup or shell 100 is less than optimal, locating a plurality of smaller diameter fasteners 120 in an acetabular cup or shell 100 having a plurality of openings 110 will operate to transmit forces from the acetabular cup or shell 100 through to the fasteners 120 and ultimately to the bone. For example, the acetabular cup or shell 100 may have a plurality of openings 110 between a range of about twenty to about sixty, or a range of about thirty to about fifty, wherein each opening 110 has a diverging angle and a smaller diameter to accept smaller fasteners 120.

Referring to FIGS. 5 through 10, a bearing insert 200 is illustrated and will now be discussed. The bearing insert 200 may be manufactured from any suitable material that is known or that may become known in the art for use within a patient's body. Such materials may comprise polyethylene, any suitable metallic material or alloy, ceramic, diamond, or any other suitable material or combination of materials for use as a bearing insert. It will be appreciated that the choice of material is dependent upon the surgeon and the desired result or application. The system of the disclosure allows a surgeon more selection and freedom to select a desired material for the insert 200 and the surgeon is not dependent upon a pre-manufactured kit or insert, including whatever mechanical lock feature that may be used to secure the insert 200 to the cup or shell 100. Thus, the disclosure contemplates allowing the surgeon to utilize any desired bearing insert 200 made of any suitable material without regard to any mechanical attachment to the acetabular cup or shell 100.

Referring specifically to FIGS. 5, 7 and 8, the disclosure may utilize a fixation material or bonding agent, such as bone cement 250, to bond the bearing insert 200 to the acetabular cup or shell 100 as the attachment mechanism between the bearing insert 200 and shell 100. It will be appreciated that the bone cement 250 may be any bonding agent or material used in the art or that may be used in the future in the art without departing from the scope of the disclosure.

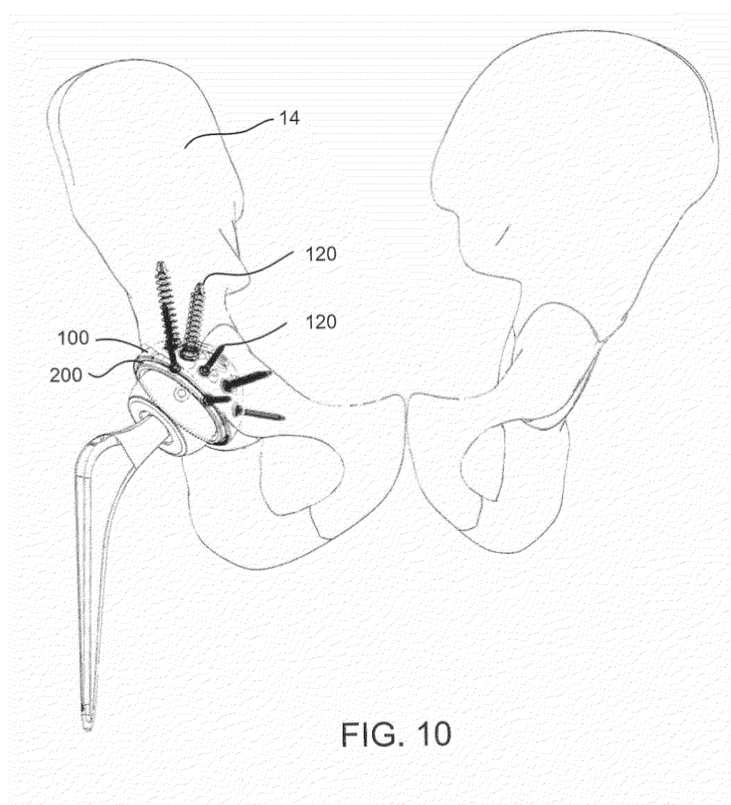

As illustrated best in FIG. 5, the bearing insert 200 may comprise an outer surface 210 and an inner surface 220. The outer surface 210 of the bearing insert 200 may be prepared with a texture or rough finish to receive or bond to the fixation material or bonding agent 250, such as bone cement. Accordingly, the outer surface 210 may comprise a non-smooth surface. In an embodiment, the texture of the outer surface 210 may be a geometry comprising a plurality of troughs or a grooves 213 or any rough or textured surface that is other than a smooth surface (illustrated best in FIG. 5). The roughened or textured outer surface 210 may be anything greater than microsized pores in order to help the fixation material or bonding agent, such as bone cement, adhere to the outer surface 210 of the bearing insert 200, and may be formed as part of or within the outer surface 210 of said bearing insert 200. Conversely, the inner surface 220 of the bearing insert 200 may be suitable for receiving therein the femoral head of the femoral component of the hip replacement (illustrated best in the image of an x-ray in FIG. 10).

For example, during use, the bone cement 250 may be placed on the outer surface 210 of the bearing insert 200. After the bone cement is applied to the bearing insert 200, the insert 200 may then be located within an interior cavity 140 of the acetabular cup or shell 100, such that the bone cement 250 that is located on the outer surface 210 of the insert 200 contacts the inner surface 102 of the cup 100 to secure the insert 200 to the cup or shell 100.

Referring back specifically to FIG. 4A, an embodiment of the system for securing an acetabular cup or shell 100 to the acetabulum may comprise a plurality of spacers 300 for receipt on the inner surface 102 of the acetabular cup 100 and specifically in the openings 110 of the cup 100. The spacers 300 may be attached or secured within the opening 110 in any manner, including a press fit, screw fit, an expandable ring or any other interlocking mechanism without departing from the scope of the disclosure.

The spacers 300 may be manufactured from the same material as the fixation material 250 used to attach or secure the bearing insert 200 to the inner surface 102 of the acetabular cup or shell 100. The spacers 300 may be used to create a mantle, such as a cement mantle, that is substantially even with respect to the thickness of the spacer's head 302, such that the insert 200 may be secured to the cup or shell 100. Further, the spacers 300, the heads 122 of the fasteners 120 and the non-smooth first or inner surface 102 of the acetabular cup 100 creates a surface that is ready to bond with cement in a manner that is sufficient to securely attach the bearing insert 200 to the acetabular cup or shell 100.

The system disclosed herein, permits a surgeon with the ability to orient the bearing insert 200 separately from the shell or cup 100. In use, as the bearing insert 200 is being oriented and positioned within the cavity 140 of the cup or shell 100, the surgeon has the ability to manipulate the insert 200 to achieve the best position for joint stability and reconstruction. The best position for locating the insert 200 to create joint stability and reconstruction may be a different position than the cup or shell 100 interfacing with the bone. Further, the system disclosed herein may reduce the need or tendency to use bone grafting material.

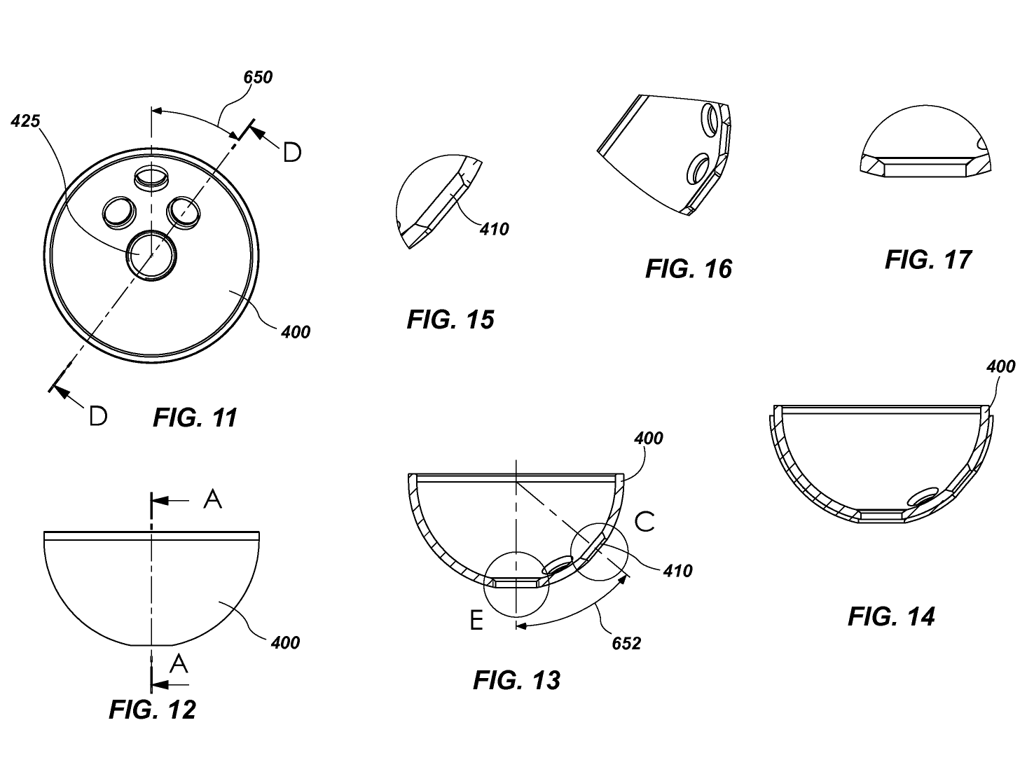

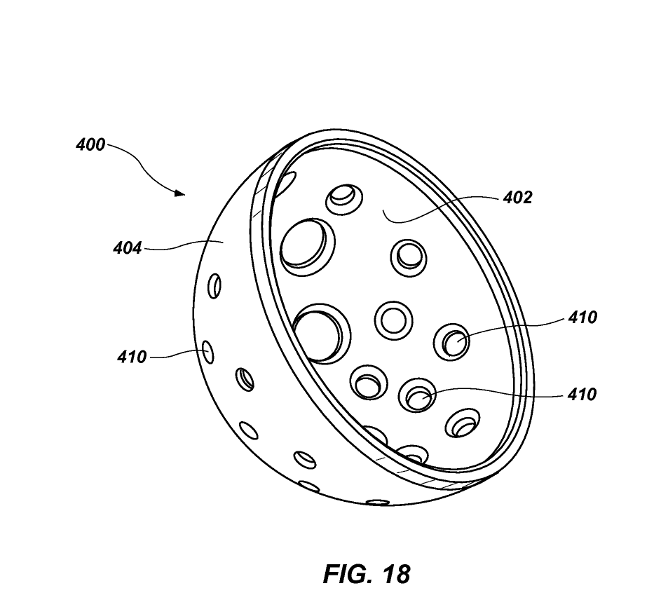

In FIGS. 11 through 22 embodiments of an acetabular cup or shell 400 are illustrated and will now be discussed. The acetabular cups or shells of FIGS. 11 through 22 may comprise the same or similar characteristics and features described above in connection with the various embodiments of the acetabular cup or shell 100 and illustrated in FIGS. 3 and 4. The following description will concentrate on the characteristics and features that have not been previously described above in connection with the various embodiments of the acetabular cup or shell 100 illustrated in FIGS. 3 and 4.

Referring to FIGS. 11 through 22, the acetabular cup or shell 400 may comprise an inner surface 402 prepared with a textured finish for receiving the bonding agent, an outer surface 404 configured to contact bone tissue, and a plurality of openings 410. Each of the openings 410 may be defined by an axis of rotation R-R (illustrated best in FIG. 22) and a sidewall 412. The axis of rotation R-R of each of the openings 410 may have differing origins, such that the plurality of openings 410 may comprise multiple origins that vary from one another. The openings 410 may comprise multiple origins 430 that vary, in which case the sidewalls 412 will be formed at varying angles (illustrated best in FIG. 4A). In other words, the sidewalls 412 may not all be oriented normal to the inner surface 402 of the cup 400. Alternatively, the plurality of openings 410 may comprise a single origin, in which case the openings may be oriented normal to the surface of the acetabular cup or shell 400 as illustrated best in FIGS. 11 through 22.

In instances in which the axis of rotation R-R of the opening 410 is normal to the inner surface 402 of the shell 400, the axis R-R may pass through an origin of the shell 400. The origin of the shell may be the central with respect to a lower lip 450 of the shell 400. It will be appreciated that some of the openings 410 may comprise an axis of rotation R-R that is angled with respect to the axis of rotation R-R or another opening 410 that is normal to the inner surface 402 of the shell 400. Such angles may vary and diverge from one opening to the next opening to allow the fasteners to be inserted at various angles and insertion points. Thus, the varying angles of the openings 410 may be oriented normal and non-normal with respect to the inner surface 402 of the shell 400, such that there are a plurality of insertion points that the plurality of fasteners are inserted into at a location where there is adequate bone.

Referring specifically to FIG. 22, the plurality of openings 410 may each comprise a counterbore 411. The counterbore 411 may comprise a radius. As noted above, the fasteners may correspond in shape and size to the openings 410 of the shell 400. Accordingly, each fastener may comprise a head having a corresponding radius to the radius of the counterbore 411, such that each fastener may be insertable into bone tissue through a corresponding opening 410 at multiple, diverging angles with respect to the axis R-R of the opening 410.

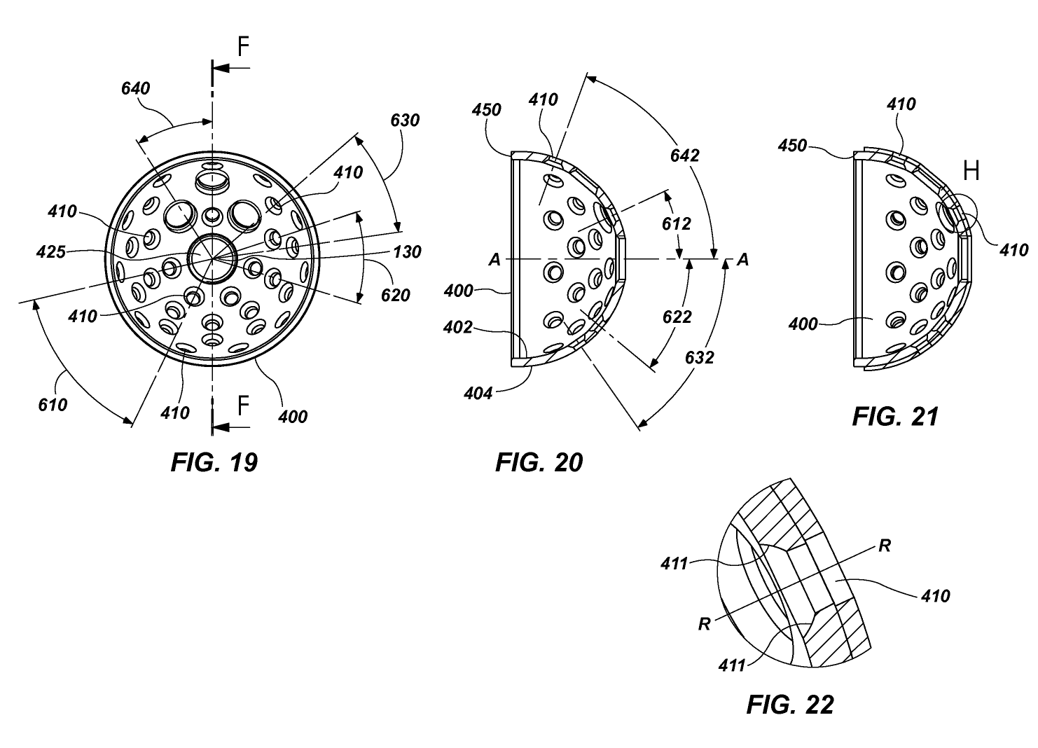

It will be appreciated that the openings 410 may be located across a portion of the acetabular cup or shell 400 or across the entire acetabular cup or shell 400 as illustrated best in FIGS. 11, 19 and 30-35. As illustrated in FIGS. 11, 19 and 30-35, it will be appreciated that the openings 410 may be spaced around the surface area of the acetabular cup or shell 400. The openings 410 may be spaced in arrays or groups, with each array or group being defined by its proximity to a central opening 425 of the acetabular cup or shell 400. The openings 410 in the same array or group are all substantially the same distance away from the central opening 425. For example, the openings 410 in the first level array or group are the closest in proximity to the central opening 425, the openings 410 in the second level array or group are the next (second overall) closest in proximity to the central opening 425, the openings 410 in the third level array or group are the next (third overall) closest in proximity to the central opening 425 and so forth. It will be appreciated that there may be one array or group or a plurality of arrays or groups of openings 410 that are formed within the acetabular cup or shell 400. For example, there may be one array, two arrays, three arrays, four arrays or more. As illustrated, the acetabular cup or shell 400 in FIG. 11 has a single array. Whereas, the acetabular cup or shell 400 in FIG. 19 has four arrays.

Referring to FIGS. 19 and 20, it will be appreciated that the openings 410 in each array level may comprise their own unique relationship to other openings 410 in the same array level. The central opening 425 may comprise a center point through which a central axis A-A of the acetabular cup or shell 400 may run. Additionally, each opening 410 may also comprise a center point 413, through which the axis of the individual opening 410 may run. The relationship between each opening 410 in each array level may be characterized by an angle, which may be formed by the intersection of the two axes at a single origin 130 of the acetabular cup or shell 400.

As illustrated best in FIG. 19, angle 610 may be formed by the intersection (at the origin 130 of the cup or shell 400) of the respective central axes of two individual openings 410 formed within the first array level and may between a range of about forty degrees and about sixty-five degrees, or between a range of about forty-five degrees and about sixty degrees, or between a range of about fifty degrees to about fifty-five degrees, such as about fifty-one degrees to about fifty-two degrees. It will be appreciated that an angle 612 formed by the intersection (at the origin 130 of the cup or shell 400) of the central axis of the acetabular cup or shell 400 and a central axis of an opening 410 within the first array level (illustrated best in FIG. 20) may be between a range of about twenty degrees and about thirty-two degrees, or between a range of about twenty-two degrees and about thirty degrees, or between a range of about twenty-three degrees to about twenty-seven degrees, such as about twenty-four degrees to about twenty-five degrees.

Additionally, as illustrated best in FIG. 19, angle 620 may be formed by the intersection (at the origin 130 of the cup or shell 400) of the respective central axes of two individual openings 410 formed within the second array level and may between a range of about twenty degrees and about fifty degrees, or between a range of about twenty-five degrees and about forty-five degrees, or between a range of about thirty degrees to about forty degrees, such as about thirty-two degrees to about thirty-seven degrees. It will be appreciated that an angle 622 formed by the intersection (at the origin 130 of the cup or shell 400) of the central axis of the acetabular cup or shell 400 and a central axis of an opening 410 within the second array level (illustrated best in FIG. 20) may be between a range of about thirty degrees and about fifty degrees, or between a range of about thirty-two degrees and about forty-eight degrees, or between a range of about thirty-five degrees to about forty-five degrees, such as about forty degrees.

Additionally, as illustrated best in FIG. 19, angle 630 may be formed by the intersection (at the origin 130 of the cup or shell 400) of the respective central axes of two individual openings 410 formed within the third array level and may between a range of about twenty degrees and about forty-five degrees, or between a range of about twenty-five degrees and about forty degrees, or between a range of about thirty degrees to about thirty-eight degrees, such as about thirty-two degrees to about thirty-three degrees. It will be appreciated that an angle 632 formed by the intersection (at the origin 130 of the cup or shell 400) of the central axis of the acetabular cup or shell 400 and a central axis of an opening 410 within the third array level (illustrated best in FIG. 20) may be between a range of about forty degrees and about seventy degrees, or between a range of about forty-five degrees and about sixty-five degrees, or between a range of about fifty degrees to about sixty degrees, such as about fifty-five degrees.

Further, as illustrated best in FIG. 19, angle 640 may be formed by the intersection (at the origin 130 of the cup or shell 400) of the respective central axes of two individual openings 410 formed within the fourth array level and may between a range of about twenty degrees and about forty-five degrees, or between a range of about twenty-five degrees and about forty degrees, or between a range of about thirty degrees to about thirty-eight degrees, such as about thirty-two degrees to about thirty-three degrees. It will be appreciated that an angle 642 formed by the intersection (at the origin 130 of the cup or shell 400) of the central axis of the acetabular cup or shell 400 and a central axis of an opening 410 within the fourth array level (illustrated best in FIG. 20) may be between a range of about fifty-five degrees and about eighty-five degrees, or between a range of about sixty degrees and about eighty degrees, or between a range of about sixty-five degrees to about seventy-five degrees, such as about seventy degrees.

Referring to FIGS. 11 and 13, angle 650 may be formed by the intersection (at the origin 130 of the cup or shell 400) of the respective central axes of two individual openings 410 formed within the cup or shell 400 and may between a range of about twenty-five degrees and about forty-five degrees, or between a range of about thirty degrees and about forty-two degrees, or between a range of about thirty-two degrees to about forty degrees, such as about thirty-five degrees to about thirty-eight degrees. It will be appreciated that an angle 652 formed by the intersection (at the origin 130 of the cup or shell 400) of the central axis of the acetabular cup or shell 400 and a central axis of an opening 410, as illustrated best in FIG. 13, may be between a range of about thirty-five degrees and about sixty-five degrees, or between a range of about forty degrees and about sixty degrees, or between a range of about forty-five degrees to about fifty-five degrees, such as about fifty degrees.

Referring now to FIGS. 11, 19, 23-35, it will be appreciated that the hip bone may be comprised of vascular safe zones, which are ideal and optimal for the placement of fasteners into the bone. The posterior-superior safe zone of the hip bone is the ideal location for the placement of fasteners due to the amount of bone present and relative absence of nuerovascular structures. The placement of fasteners into the posterior-superior safe zone aids in stabilizing the shell within the acetabular cavity. Hence, the posterior-superior safe zone is the best-suited location for inserting fasteners into the hip bone and results in the lowest risk of implant failure.

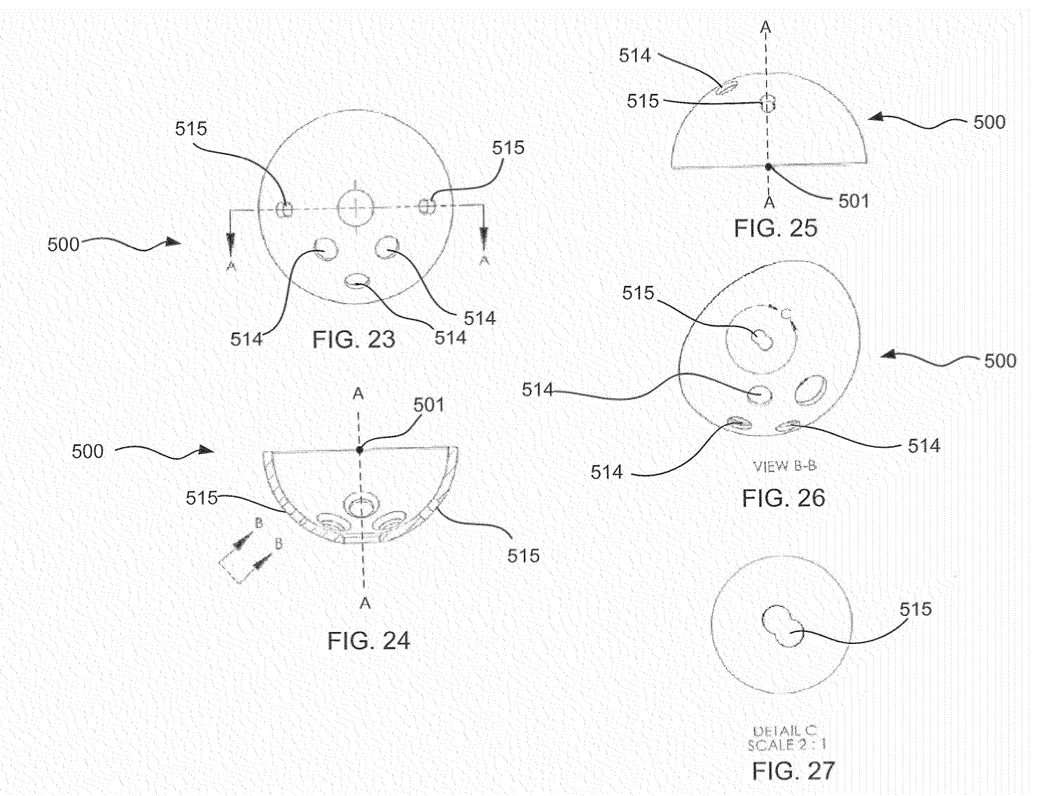

The implant illustrated in FIGS. 23-29 may comprise any combination or all of the features discussed above in connection with other embodiments of the shell, fasteners and bearing insert. For the sake of convenience, only the differences between FIGS. 23-29 and the other embodiments disclosed herein will be primarily addressed. The implant illustrated in FIGS. 23-29 may generally comprise a shell 500, fasteners 520a and 520b, and a bearing insert 550.

The shell 500 may comprise an origin or radiant 501, an inner surface 502, an outer surface 504 and a plurality of openings 510 formed through the inner surface 502 and the outer surface 504. The origin or radiant 501 may be defined as a point located on the central axis A-A of the shell 500, about which the shell 500 may conceivably rotate such that the shell may be considered substantially symmetrical with respect to the axis and origin or radiant 501 as illustrated in FIGS. 23 through 25. The shell 500 may comprise a spatial concentration of openings 510 that may be within a range of about five to about fifteen openings per concentration area of the shell 500, wherein the concentration area is defined as a surface area of the outer surface 504 of the shell 500 that may be between a range of about eighty square millimeters to about one-hundred and seventy-five square millimeters. It will be appreciated that the concentration area may or may not be the entire surface area of the outer surface 504 of the shell 500. For example, the spatial concentration of openings per concentration area may be concentrated into one or more quadrants of the shell 500 or may be distributed across the entire surface area of the outer surface 504 of the shell 500 without departing from the scope of the disclosure.

The plurality of openings 510 may each be sized, shaped, configured and dimensioned for receiving a fastener 520 therein. The openings 510 may comprise at least two sizes, a first size opening 514 configured and dimensioned to receive the first or primary fasteners 520a therein and a second size opening 516 configured and dimensioned to receive the second or secondary fasteners 520b therein.

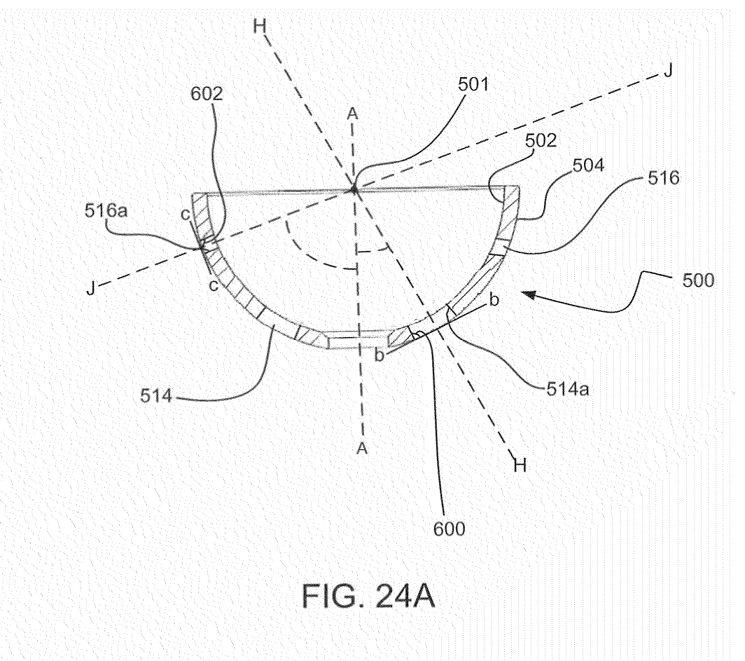

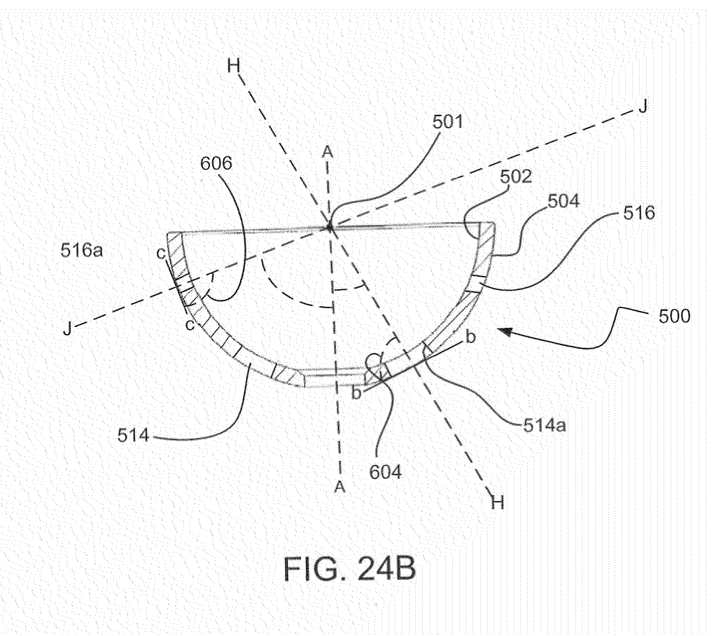

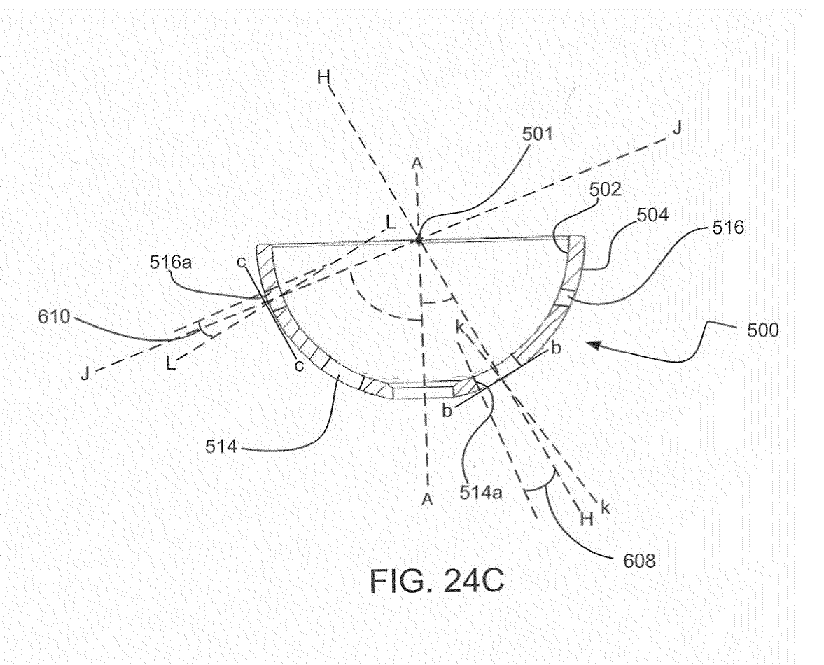

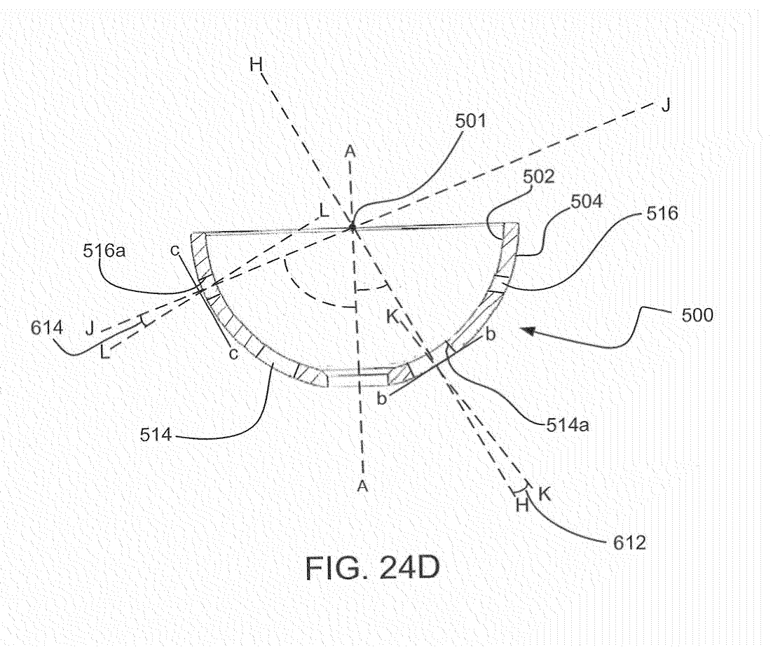

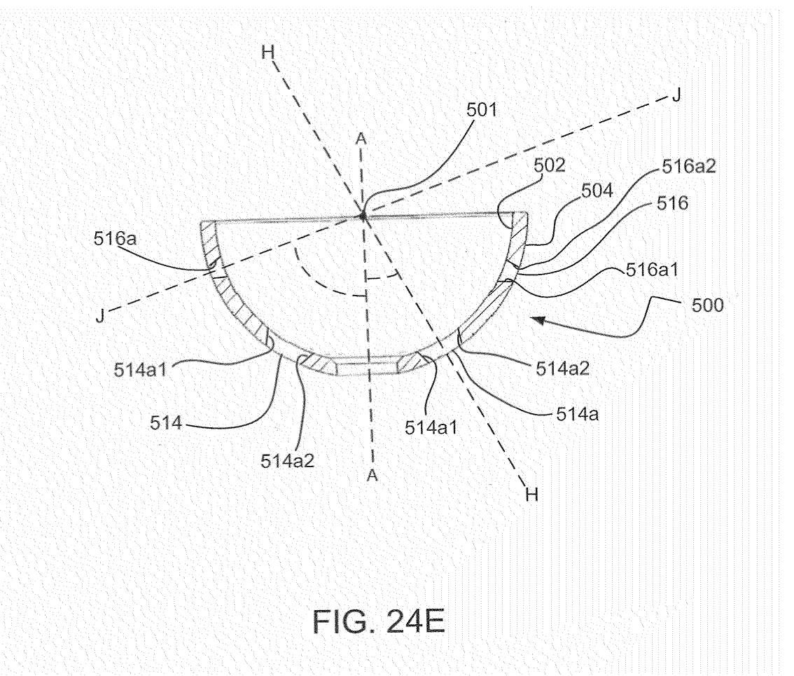

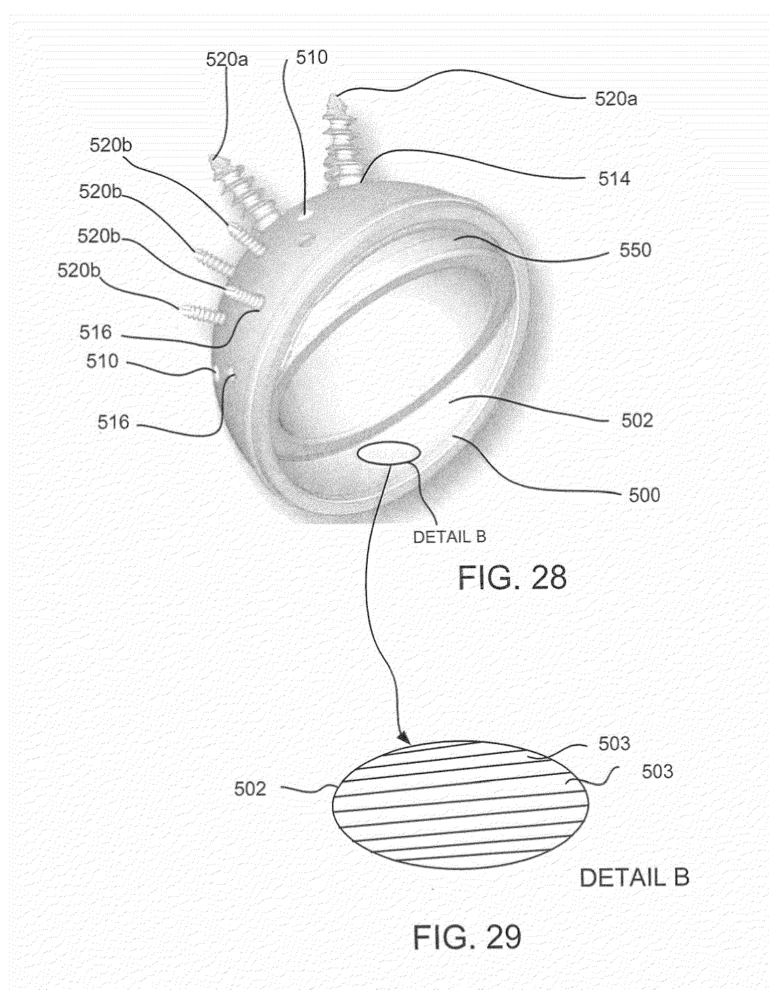

Referring specifically to FIGS. 24A and 28, the shell 500 may comprise a plurality of first openings 514 that each have a diameter that corresponds with the diameter of a first fastener 520a, such that the first fastener 520a may be inserted thereinto. These first openings 514 may be distributed over the shell 500 in order to target the safe zones of the hip bone, such as the posterior-superior zone. The first openings 514 may be formed at an angle θ with respect to the central the axis A-A of the shell 500, which passes through the origin 501. Angle θ may be within a range of about twenty-five degrees to about forty degrees in order to receive and position a first fastener in posterior-superior zone as shown in FIGS. 24A and 28.

In an embodiment, the first openings 514 may be formed at an angle with respect to the central axis A-A of the shell 500. In an embodiment one or more of the first openings 514 may be defined by a sidewall 514a that may form an acute angle with a line that is tangent to an exterior surface 504 of the shell 500 and that intersects a central axis H-H of the first opening 514 defined by the sidewall 514a. The angle may be within a range of about twenty-five degrees to about forty degrees.

In an embodiment one or more of the first openings 514 may be defined by a sidewall 514a that defines a central axis H-H of the first opening 514 and that may form an acute angle with respect to a line that may be both tangent to an exterior surface 504 of the shell 500 and that intersects the central axis H-H of the first opening 514. The angle may be within a range of about twenty-five degrees to about forty degrees.

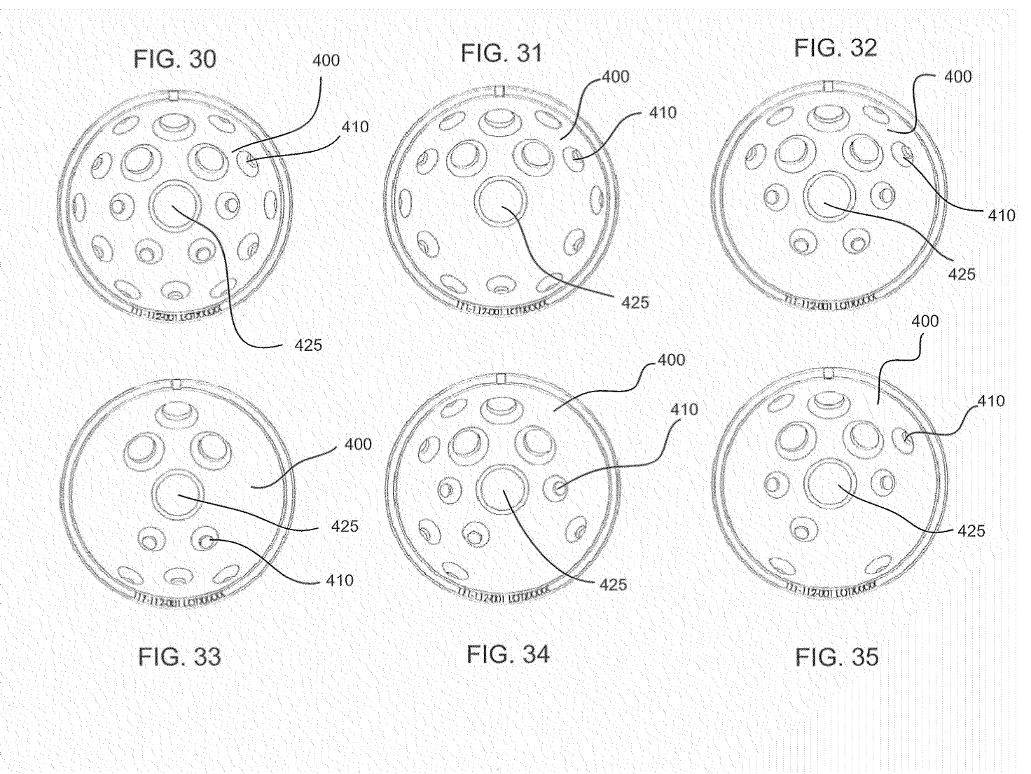

The shell 500 may also comprise a plurality of second openings 516, which may be smaller than the first openings 514. The second openings 516 may be distributed around the shell 500, whether equally around the entire four quadrants of the shell 500 or concentrated in one or more particular quadrants of the shell 500 (illustrated best in FIGS. 30-35), such that when the second fasteners 520b are inserted thereinto the second fasteners are insertable into the bone in a variety of directions and at diverging angles. In an embodiment, the axis of the second openings 516 may be normal or perpendicular to the tangent of the curve of the inner surface 502 or outer surface 504 of the shell 500. In an embodiment, the second openings 516 may have diverging angles, where the central axis J-J of the second opening 516 may be formed at an angle with respect to the central axis A-A of the shell 500 that passes through the origin 501, wherein the angle may vary between about twenty degrees to about eighty degrees as shown in FIG. 24A.

In an embodiment, the second openings 516 may be formed at an angle with respect to the central axis A-A of the shell 500. In an embodiment, one or more of the second openings 516 may be defined by a sidewall 516a that may form an acute angle with a line that is tangent to an exterior surface 504 of the shell 500 and that may intersect the central axis J-J of the second opening 516 defined by the sidewall 516a.

In an embodiment one or more of the second openings 516 may be defined by a sidewall 516a that defines the central axis J-J of the second opening 516 and that may form an acute angle with a line that is both tangent to an exterior surface 504 of the shell 500 and that intersects the central axis J-J.

Referring now to FIG. 24A, in an embodiment, one or more of the plurality of openings, whether first openings 514 or second openings 516, may be defined by a sidewall 514a or 516a that may form an acute angle (600 or 602) with a line (b-b or c-c) that is tangent to an exterior surface 504 of the shell 500 and that may intersect a central axis H-H or J-J of the opening 514 or 516 defined by the sidewall 514a or 516a.

Referring now to FIG. 24B, in an embodiment, one or more of the plurality of openings, whether first openings 514 or second openings 516, may be defined by a sidewall 514a or 516a that defines a central axis H-H or J-J of the opening 514 or 516, which central axis H-H or J-J may form a right angle or an acute angle (604 or 606) with a line (b-b or c-c) that is both tangent to an exterior surface 504 of the shell 500 and that may intersect the central axis H-H or J-J of the opening 514 or 516.

Referring now to FIG. 24C, in an embodiment, one or more of the plurality of openings, whether first openings 514 or second openings 516, may be defined by a sidewall 514a or 516a that may form an acute angle (608 or 610) with an imaginary line (K-K or L-L) that is perpendicular to a tangent line (b-b or c-c) of an exterior surface 504 of the shell 500, wherein the tangent line (b-b or c-c) intersects a central axis H-H or J-J of the opening 514 or 516 defined by the sidewall 514a or 516a. It will be appreciated that the imaginary line may be co-axial with respect to the central axis H-H or J-J of the opening 514 or 516.

Referring now to FIG. 24D, in an embodiment, one or more of the plurality of openings, whether first openings 514 or second openings 516, may be defined by a sidewall 514a or 516a that defines a central axis H-H or J-J of the opening 514 or 516, which central axis H-H or J-J may form an acute angle (612 or 614) with an imaginary line (K-K or L-L) that is perpendicular to a line (b-b or c-c) that is both tangent to an exterior surface 504 of the shell 500 and that intersects the central axis H-H or J-J of the opening 514 or 516.

Referring now to FIG. 24E, in an embodiment, one or more of the plurality of openings, whether first openings 514 or second openings 516, may be defined by a sidewall 514a or 516a that defines a central axis H-H or J-J of the opening 514 or 516. The sidewall 514a or 516a may include a first opposing side-line 514a1 or 516a1 and a second opposing side-line 514a2 or 516a2. The first opposing side-line (514a1 or 516a1) and the second opposing sideline (514a2 or 516a2), respectively, may reside on opposing sides of the central axis H-H or J-J of the opening 514 or 516, such that each sideline 514a1, 514a2 or 516a1, 516a2 progresses toward the central axis H-H or J-J of the opening 514 or 516 in an interior-to-exterior direction (e.g., progresses from the interior surface 502 toward the exterior surface 504) with respect to the shell 500, such that the entrance to the opening at the interior portion of the shell is larger than the exit to the opening at the exterior portion of the shell.

It will be appreciated that the central axis H-H or J-J of the opening 514 or 516 may be defined as extending through the center of the opening 514 or 516. For example, in the case of an opening that may be cylindrical or circular in shape, the central axis extends through the center point of the cylinder or circle, which center point may be defined by the radius of the cylinder or circle, such that the central axis passes through the center of the opening.

Referring now to FIGS. 23-27, it will be appreciated that the rim area of the shell 500 may be an area where diverging openings may be particularly useful. Further, it will be appreciated that an embodiment may comprise one or more figure 8 (eight) shaped openings 515, where the opening 515 is defined by the overlapping circumference of two openings as illustrated best in FIGS. 23-27. The figure 8 shape may be used in a substantially hemispherical shell and may provide a surgeon with the ability to insert fasteners 520a or 520b at different and varying angles in order to locate fasteners 520 as securely as possible in the bone, depending upon bone anatomy of the patient, during a procedure.

Referring specifically now to FIG. 28, the plurality of first fasteners 520a may each have a diameter that is greater than or equal to about 6.0 millimeters. For example, in an embodiment the diameter of the first fastener 520a may be about 6.5 millimeters. The number of first fasteners 520a used in a case may change depending upon the amount and location of available bone. Thus, the number of first fasteners 520a used in a given case may be within a range of about two to about six fasteners. The length of the first fasteners 520a may be within a range of about thirty millimeters to about forty-five millimeters or longer without departing from the scope of the disclosure. These larger fasteners, which may be screws or pins or other fasteners to secure an implant to bone, may be used to provide stability within the bone. These larger, first fasteners 520a may be used to locate and secure the shell in the posterior-superior safe zone of the hip bone.

Conversely, the plurality of second fasteners 520b may each have a diameter that is less than or equal to 3.5 millimeters for securing said shell to bone tissue. The second fasteners 520b may be inserted into any location where there is adequate bone available and is not dependent upon a large amount of bone due to their smaller size. The second fasteners 520b may be inserted in a bony area that may be viewed as deficient or otherwise inadequate to receive the larger, first fasteners 520a. For example, in an embodiment the diameter of the second fastener 520b may be about 2.7 millimeters.

The number of second fasteners 520b used in a given case may change depending upon the amount and location of available bone. In most situations, the larger number of second fasteners 520b used the more fixation points there will be to secure the shell 500 to the bone, such that forces may be distributed through each fixation point to the bone. Thus, the number of second fasteners 520b used in a given case may be at least twelve in number and may be within a range of about twelve to about thirty or more in number.

In an embodiment, the plurality of second fasteners 520b may be at least twelve fasteners in number. In an embodiment, the plurality of second fasteners 520b may be at least fifteen fasteners in number. In an embodiment, the plurality of second fasteners 520b may be at least twenty fasteners in number. In an embodiment, the plurality of second fasteners 520b may be at least twenty-five fasteners in number. In an embodiment, the plurality of second fasteners 520b may be at least thirty fasteners in number. In an embodiment, the plurality of second fasteners 520b may be at least thirty-five fasteners in number. In an embodiment, the plurality of second fasteners 520b may be at least forty fasteners in number. In an embodiment, the plurality of second fasteners 520b may be at least forty-five fasteners in number. In an embodiment, the plurality of second fasteners 520b may be at least fifty fasteners in number. In an embodiment, the plurality of second fasteners 520b may be at least fifty-five fasteners in number. In an embodiment, the plurality of second fasteners 520b may be about fifteen fasteners to about sixty fasteners in number.

It will be appreciated that the number of first and second fasteners 520b may be dependent on the bone quality and whether and to what extent there is bone deficiency. The number of second fasteners 520b may increase to provide added stability and load transfer. The more unstable the bone is, the more important it is to locate as many second fasteners 520b as possible into that bone, since the larger first fasteners 520a may be more difficult to secure to that deficient bone. The disclosure contemplates using as many secondary fasteners 520b as possible to provide as many fixation points as possible, no matter what the quality of the bone is. As the number of second fasteners 520b secured to bone increases, the more fixation points there are, which results in providing added stability and load distribution through those fixation points. The result may be an even distribution of force placed on the implant and a longer implant life.

It will be appreciated that each of the plurality of second fasteners 520b may comprise a length that is less than thirty millimeters, and may be between about twenty millimeters to about twenty-five millimeters in length. Thus, the length of the second fasteners 520b may be less than the average length of an acetabular screw or pin, which is typically within a range of about thirty-five to forty-five millimeters.

It will be appreciated that each of the plurality of first fasteners 520a and the plurality of second fasteners 520b may be insertable through one of the first or second openings 514 and 516, respectively, of the shell 500 and into the bone at an angle with respect to the origin of said shell 500. The result is to distribute and transfer forces placed on shell 500 to the bone at multiple, varying insertion points, which correspond with the fastener 520 inserted into the bone.

Referring to FIGS. 28 and 29, the inner surface 502 of the shell 500 may be textured or otherwise prepared for receiving the fixation material or bonding agent, such as bone cement. In an embodiment, the texture of the inner surface 502 may be a geometry comprising a plurality of troughs or a grooves 503 or any rough or textured surface that is other than a smooth surface (illustrated best in FIG. 29). The roughened or textured inner surface 502 may be anything greater than microsized pores in order to help the fixation material or bonding agent, such as bone cement, adhere to the inner surface 502 of the shell 500, and may be formed as part of or within the inner surface 502 of the shell 500.

Those having ordinary skill in the relevant art will appreciate the advantages provided by the features of the disclosure. For example, it is a potential feature to provide a low stress, multiple fixation acetabular component for use in surgical situations where there is inadequate bone for traditional fixation. It is another potential feature to use multiple fasteners that may be inserted into the bone at diverging angles to provide maximum fixation in the bone. It is another potential feature to insert about twelve to about sixty fasteners into the bone at diverging angles. It is another potential feature to provide a plurality of openings or holes in an acetabular component at diverging angles to thereby permit a plurality of fasteners having a length that is less than thirty millimeters, and may be between about twenty millimeters to about twenty-five millimeters, in length to be inserted therethrough. It is another potential feature to provide an acetabular component that distributes stress over a larger area than traditional methods, resulting in less stress due to the number of fasteners inserted into the bone. It is another potential feature to provide an acetabular component with multiple fixation angles.

In the foregoing Detailed Description, various features of the disclosure are grouped together in a single embodiment for the purpose of streamlining the disclosure. This method of disclosure is not to be interpreted as reflecting an intention that the claimed disclosure requires more features than are expressly recited in each claim. Rather, inventive aspects lie in less than all features of a single foregoing disclosed embodiment.

It is to be understood that the above-described arrangements are only illustrative of the application of the principles of the disclosure. Numerous modifications and alternative arrangements may be devised by those skilled in the art without departing from the spirit and scope of the disclosure and the disclosure is intended to cover such modifications and arrangements. Thus, while the disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A system for securing an orthopedic implant to a bone, comprising:
a shell comprising a central axis, an inner surface, an outer surface and a plurality of openings formed therethrough, wherein the shell comprises a spatial concentration of openings in a range of about five to about fifteen openings per concentration area of said shell, wherein said concentration area is a surface area of said outer surface of said shell that is between a range of about eighty square millimeters to about one-hundred and seventy-five square millimeters;
a plurality of first fasteners and a plurality of second fasteners, wherein each of the first fasteners has a diameter of that is greater than or equal to 6.0 millimeters wherein each of the plurality of second fasteners has a diameter that is less than or equal to 3.5 millimeters for securing said shell to bone tissue;
wherein each of the plurality of second fasteners comprises a length that is less than thirty millimeters;
wherein each of the plurality of first fasteners and the plurality of second fasteners is insertable through one of the openings of said shell and into the bone at an angle with respect to the central axis of said shell, thereby distributing and transferring forces placed on said shell to the bone at multiple, varying insertion points, which correspond with the fastener inserted into the bone.

2. The system of claim 1, wherein the spatial concentration of openings per concentration area is distributed across the entire surface area of the outer surface of the shell.

3. The system of claim 1, wherein the plurality of second fasteners comprise at least twelve fasteners.

4. The system of claim 1, wherein the plurality of second fasteners comprise at least fifteen fasteners.

5. The system of claim 1, wherein the plurality of second fasteners comprise at least twenty fasteners.

6. The system of claim 1, wherein the plurality of second fasteners comprise at least twenty-five fasteners.

7. The system of claim 1, wherein the plurality of second fasteners comprise at least thirty fasteners.

8. The system of claim 1, wherein the plurality of second fasteners comprise at least thirty-five fasteners.

9. The system of claim 1, wherein the plurality of second fasteners comprise at least forty fasteners.

10. The system of claim 1, wherein the plurality of second fasteners comprise at least forty-five fasteners.

11. The system of claim 1, wherein the plurality of second fasteners comprise at least fifty fasteners.

12. The system of claim 1, wherein the plurality of second fasteners comprise at least fifty-five fasteners.

13. The system of claim 1, wherein the plurality of second fasteners comprise about fifteen fasteners to about sixty fasteners.

14. The system of claim 1, wherein each of the plurality of second fasteners are insertable into the bone at an angle that varies and diverges from another one of the plurality of second fasteners, such that there is a fixation point where each of the plurality of fasteners inserted into bone, which allows forces to be distributed and transferred from the shell to the plurality of second fasteners inserted into the bone at multiple, varying insertion points and from the plurality of second fasteners to the bone itself, and wherein a length of each second fastener is within a range of about twenty millimeters to about twenty-five millimeters.

15. The system of claim 1, wherein the plurality of fasteners comprise an average diameter that is between a range of about three millimeters to about five millimeters and an average length that is between a range of about twenty-five millimeters to about thirty-five millimeters in length.

16. The system of claim 1, wherein each of the openings comprise a diameter that corresponds with the diameter of a corresponding fastener.

17. The system of claim 16, wherein the plurality of openings comprise an average diameter that is same as the average diameter of the plurality of fasteners.

18. The system of claim 1, wherein at least one of the openings is shaped as a FIG. 8 and is defined by an overlapping circumference of two adjacent openings.

19. The system of claim 18, wherein the shell is substantially hemispherical, such that the FIG. 8 shaped opening in the substantially hemispherical shell provides an ability to insert a plurality of fasteners at different and varying angles to thereby locate said fasteners as securely as possible in the bone, depending upon bone anatomy, during a procedure.

20. The system of claim 1, wherein the inner surface of the shell is textured to thereby receive a bonding agent therein.

21. The system of claim 20, wherein the texture is formed by a plurality of troughs formed in the inner surface of the shell.

22. The system of claim 20, wherein the texture is formed by a plurality of grooves formed in the inner surface of the shell.

23. The system of claim 1, wherein the plurality of openings in the shell comprise a plurality of second openings, which have diameters that correspond with the diameters of the plurality of second fasteners, such that each of the plurality of second fasteners is insertable into each of the plurality of second openings to assist in securing the shell to the bone; and
wherein the plurality of openings in the shell further comprise a plurality of first openings, which have diameters that correspond with the diameters of the plurality of first fasteners, such that each of the plurality of first fasteners is insertable into each of the plurality of first openings to thereby anchor the shell to the bone.

24. The system of claim 23, wherein the first openings are distributed over the shell to target safe zones of the hip bone, and wherein said first openings are formed at an angle with respect to the central axis of said shell, wherein said angle is within a range of about twenty-five degrees to about forty degrees.

25. The system of claim 23, wherein each of the second openings are defined by a sidewall that defines a central axis of the second opening, and wherein an acute angle is formed by the intersection of the central axis of the second opening with a central axis of the shell.

26. The system of claim 23, wherein said second openings are formed at an angle with respect to the central axis of said shell, wherein one or more of the second openings are defined by a sidewall that forms an acute angle with a line that is tangent to an exterior surface of the shell and that intersects a central axis of the opening defined by said sidewall.

27. The system of claim 1, wherein one or more of the plurality of openings are defined by a sidewall that forms an acute angle with a line that is tangent to an exterior surface of the shell and that intersects a central axis of the opening defined by said sidewall.

28. The system of claim 1, wherein one or more of the plurality of openings are defined by a sidewall that defines a central axis of the opening, which forms an acute angle with a line that is both tangent to an exterior surface of the shell and that intersects said central axis of said opening.

29. The system of claim 1, wherein one or more of the plurality of openings are defined by a sidewall that forms an acute angle with an imaginary line that is perpendicular to a tangent line of an exterior surface of the shell, wherein said tangent line intersects a central axis of the opening defined by said sidewall.

30. The system of claim 29, wherein the imaginary line is co-axial to the central axis of the opening.

31. The system of claim 1, wherein one or more of the plurality of openings are defined by a sidewall that defines a central axis of said opening and that forms an acute angle with an imaginary line that is perpendicular to a line that is both tangent to an exterior surface of the shell and that intersects said central axis of said opening.

32. The system of claim 1, wherein one or more of the plurality of openings are defined by a sidewall that defines a central axis of said opening, wherein said sidewall includes a first opposing side-line and a second opposing side-line, and wherein said first and second opposing sidelines reside on opposing sides of the central axis of said opening and each progresses toward said central axis of said opening in an interior-to-exterior direction with respect to the shell.

33. The system of claim 1, wherein the system further comprises a plurality of spacers having a head; wherein each of the spacers is sized and shaped for insertion and securement into a vacant opening of the shell.

34. The system of claim 33, wherein the system further comprises a bonding agent and a bearing insert having an outer surface prepared for receiving the bonding agent; wherein each spacer is inserted from the inner surface of the shell into a vacant opening of said shell, such that the head of each spacer is exposed within the inner surface of the shell to create a mantle of the bonding agent to secure the outer surface of the bearing insert to the inner surface of the shell.

35. The system of claim 1, wherein the system further comprises a bearing insert and a bonding agent;
wherein the spatial concentration of openings per concentration area is distributed across the entire surface area of the outer surface of the shell;
wherein the plurality of second fasteners comprises between about twelve fasteners and about sixty fasteners;
wherein each of the plurality of second fasteners are insertable into the bone at an angle that varies and diverges from another one of the plurality of second fasteners, such that there is a fixation point where each of the plurality of fasteners inserted into bone, which allows forces to be distributed and transferred from the shell to the plurality of second fasteners inserted into the bone at multiple, varying insertion points and from the plurality of second fasteners to the bone itself;
wherein a length of each second fastener is within a range of about twenty millimeters to about twenty-five millimeters;
wherein the plurality of fasteners comprise an average diameter that is between a range of about three millimeters to about five millimeters and an average length that is between a range of about twenty-five millimeters to about thirty-five millimeters in length;
wherein each of the openings comprise a diameter that corresponds with the diameter of a corresponding fastener;
wherein the plurality of openings comprise an average diameter that is same as the average diameter of the plurality of fasteners;
wherein at least one of the openings is shaped as a figure 8 and is defined by an overlapping circumference of two adjacent openings;
wherein the shell is substantially hemispherical, such that the figure 8 shaped opening in the substantially hemispherical shell provides an ability to insert a plurality of fasteners at different and varying angles to thereby locate said fasteners as securely as possible in the bone, depending upon bone anatomy, during a procedure;
wherein the inner surface of the shell is textured to thereby receive the bonding agent therein;
wherein the bearing insert has a prepared outer surface that is textured to receive the bonding agent, wherein the bearing insert is securable to the inner surface of the shell via the bonding agent;
wherein the system further comprises a plurality of spacers having a head;
wherein each of the spacers is sized and shaped for insertion and securement into a vacant opening of the shell; and
wherein each spacer is inserted from the inner surface of the shell into a vacant opening of said shell, such that the head of each spacer is exposed within the inner surface of the shell to create a mantle of the bonding agent to secure the outer surface of the bearing insert to the inner surface of the shell.

36. A system for securing an orthopedic implant to a bone, comprising:
a bonding agent configured to attach components of the system together;
a shell comprising an outer surface configured to contact bone tissue, an inner surface prepared with a textured finish for receiving the bonding agent, and a plurality of openings formed between the outer surface and the inner surface, wherein each of the openings is defined by an axis of rotation;
a plurality of fasteners, wherein each faster is insertable through one of the openings and into bone tissue at varying, divergent angles with respect to the axis of rotation of each opening in a splayed manner to distribute and transfer forces placed on the shell to the bone tissue at multiple, varying angles over a large area of the bone; and
a bearing insert having a prepared outer surface to receive the bonding agent, wherein the bearing insert is securable to the inner surface of the shell via the bonding agent.

37. The system of claim 36, wherein the axis of rotation of each of the openings has an origin, and wherein the origins differ, such that the shell comprises multiple origins that vary from one another, wherein the varying angles of the openings comprise normal and non-normal angles with respect to the inner surface of the shell, such that there are a plurality of insertion points that the plurality of fasteners are inserted into at a location where there is adequate bone.

38. The system of claim 37, wherein the axes of rotation of the plurality of openings are formed at angles with respect to the inner surface of the shell, wherein the angles diverge from each other.

39. The system of claim 36, wherein the system further comprises a plurality of spacers having a head; wherein each of the spacers is sized and shaped for insertion and securement into a vacant opening of the shell.

40. The system of claim 39, wherein each spacer is inserted from the inner surface of the shell into a vacant opening of said shell, such that the head of each spacer is exposed within the inner surface of the shell to create a mantle of the bonding agent to secure the outer surface of the bearing insert to the inner surface of the shell.

41. The system of claim 36, wherein the plurality of fasteners and the plurality of openings are sized and shaped to correspond with each other.

42. The system of claim 41, wherein each of the plurality of openings comprise a counterbore having a radius; and wherein the plurality of fasteners each comprise a head having a corresponding radius, such that each fastener is insertable into bone tissue through a corresponding opening at multiple, diverging angles with respect to the axis of the opening.

43. The system of claim 36, wherein the plurality of openings in the shell range in number between twenty and sixty.

44. The system of claim 36, wherein the inner surface of the shell has a finish for receiving the bonding agent that is a non-smooth finish comprising hydroxyapatite.

45. The system of claim 36, wherein the plurality of openings each have a counterbore having a radius.

46. The system of claim 36, wherein the plurality of openings each have a diameter, wherein the diameters vary from opening to opening.

47. The system of claim 36, wherein the outer surface of the bearing insert has a finish for receiving bonding agent that is a non-smooth finish comprising hydroxyapatite.

48. The system of claim 36, wherein the inner surface of the bearing insert is configured and dimensioned for receiving a femoral head of a femoral component therein.

49. The system of claim 46, wherein the fasteners comprise diameters that are sized and shaped to correspond with the size and shape of diameters of the openings of the shell.

50. The system of claim 36, wherein the fasteners have diameters within a range of about 2.7 millimeters to about 6.5 millimeters; and wherein the fasteners have a length that is within a range of about twenty to forty-five millimeters, such that the plurality of fasteners operate together to spread load over a large area of bone tissue to reduce stress concentration in any one area.

51. The system of claim 39, wherein the spacers are manufactured from the same material as the bonding agent.

52. The system of claim 40, wherein the mantle that is created is of a substantially even thickness, which corresponds to a thickness of the exposed heads of the spacers.

53. The system of claim 39, wherein each of the spacers is secured within a corresponding opening of the shell via an interlocking mechanism.

54. The system of claim 53, wherein the interlocking mechanism is a press fit, a screw fit, or an expandable ring.

55. An orthopedic implant for securing to a bone, comprising:
a shell comprising an outer surface configured to contact bone tissue, an inner surface, and a plurality of openings each of which has an axis;
wherein each of the axes comprises an angle that is formed with respect to a line that is tangent to a curvature of the inner surface of the shell at the opening;
wherein the each of the axes are formed at divergent angles with respect to each other; and
a plurality of fasteners, wherein each faster is insertable through one of the openings and into bone tissue at varying divergent angles in a splayed manner to distribute and transfer forces placed on the shell to the bone tissue at multiple, varying angles and over a larger area of the bone.

56. The orthopedic implant of claim 55, wherein each of the axes has a different origin with respect to another opening, such that the openings comprise multiple and varying origins; and wherein the angles of the axes comprise at least one normal angle and at least one non-normal angle with respect to the line tangent to the curvature of the inner surface of the shell, such that there are a plurality of insertion points that the plurality of fasteners are inserted into at a location where there is adequate bone.

57. The orthopedic implant of claim 55, wherein the system further comprises a plurality of spacers having a head; wherein each of the spacers is sized and shaped for insertion and securement into a vacant opening of the shell.

58. The orthopedic implant of claim 57, wherein each spacer is inserted from the inner surface of the shell into a vacant opening of said shell, such that the head of each spacer is exposed within the inner surface of the shell to create a mantle of a bonding agent.

59. The orthopedic implant of claim 55, wherein the plurality of fasteners and the plurality of openings are sized and shaped to correspond with each other.

60. The orthopedic implant of claim 59, wherein each of the plurality of openings comprise a counterbore having a radius; and wherein the plurality of fasteners each comprise a head having a corresponding radius, such that each fastener is insertable into bone tissue through a corresponding opening at multiple, diverging angles with respect to the axis of the opening.

61. The orthopedic implant of claim 55, wherein the plurality of openings in the shell range in number between twenty and sixty.

62. The orthopedic implant of claim 55, wherein the inner surface of the shell has a finish for receiving a bonding agent, wherein the finish is a non-smooth finish comprising hydroxyapatite.

63. The orthopedic implant of claim 55, wherein the plurality of openings each have a counterbore having a radius.

64. The orthopedic implant of claim 55, wherein the plurality of openings each have a diameter, wherein the diameters vary from opening to opening.

65. The orthopedic implant of claim 55, wherein the system further comprises a bearing insert having an outer surface; wherein the outer surface of the bearing insert has a finish for receiving a bonding agent, wherein the finish is a non-smooth finish comprising hydroxyapatite.

66. The orthopedic implant of claim 65, wherein the inner surface of the bearing insert is configured and dimensioned for receiving a femoral head of a femoral component therein.

67. The orthopedic implant of claim 64, wherein the fasteners comprise diameters that are sized and shaped to correspond with the size and shape of diameters of the openings of the shell.

68. The orthopedic implant of claim 55, wherein the fasteners have diameters within a range of about 2.7 millimeters to about 6.5 millimeters; and wherein the fasteners have a length that is within a range of about twenty to about forty-five millimeters, such that the plurality of fasteners operate together to spread load over a large area of bone tissue to reduce stress concentration in any one area.

69. The orthopedic implant of claim 58, wherein the spacers are manufactured from the same material as the bonding agent.

70. The orthopedic implant of claim 58, wherein the mantle that is created is of a substantially even thickness, which corresponds to a thickness of the exposed heads of the spacers.

71. The orthopedic implant of claim 57, wherein each of the spacers is secured within a corresponding opening of the shell via an interlocking mechanism.

72. The orthopedic implant of claim 71, wherein the interlocking mechanism is a press fit, a screw fit, or an expandable ring.

73. A method of securing an orthopedic implant to a bone, comprises:
preparing the bone to receive a shell, wherein the shell comprises a plurality of openings each of which is defined by a sidewall, wherein each sidewall is formed at an angle with respect to an inner surface of said shell, and wherein the sidewalls of the openings are formed at divergent angles with respect to each other;
inserting a plurality of fasteners into the bone, wherein each fastener is inserted through one of the openings and into the bone;
applying a bonding agent to a prepared outer surface of a bearing insert;
securing the bearing insert to the shell with the bonding agent, such that the bearing insert is located in a position for joint stability and reconstruction;
wherein the bearing insert is positioned at a different angle than the shell interfacing the bone.
